(19) United States
(12) Patent Application Publication
(10) Pub. No.: US 2015/0360079 A1
(43) Pub. Date: Dec. 17, 2015

(54) PULMONARY HARMONICA DEVICE AND METHOD OF USING A PULMONARY HARMONICA DEVICE

(71) Applicant: Halcyon Research, Inc. DBA Harmonica Techs, Sarasota, FL (US)

(72) Inventors: Dana Kesslen Keller, Sarasota, FL (US); Mary Lou Casadevall-Keller, Sarasota, FL (US)

(21) Appl. No.: 14/409,883

(22) PCT Filed: Jul. 15, 2014
(86) PCT No.: PCT/US14/46641

§ 371 Date: Dec. 19, 2014

Related U.S. Application Data
(60) Provisional application No. 61/856,283, filed on Jul. 19, 2013.

(51) Int. Cl. A63B 23/18; G10D 7/12; A61M 16/00

(52) U.S. Cl. CPC A63B 23/18; A61M 16/0006; G10D 7/123

(57) ABSTRACT

The present invention is a pulmonary harmonica for achieving health and wellness through controlled breathing. The invention is a modified harmonica with specifically low-tuned reeds that produce frequencies not associated with normal musical performance that allow the user to generate a stream of controlled low frequency sound waves that break up congestion in the user's lungs and sinuses. The invention includes a pulmonary rehabilitation treatment using inspiratory and expiratory muscle training that encourages continued use of the pulmonary harmonica so that use of the device and diaphragmatic breathing become habitual through practice.

### CROSS REFERENCE TO RELATED APPLICATIONS

This application claims the benefit of and priority to International Application No. PCT/US2014/046641, filed Jul. 15, 2014, and U.S. Provisional Application No. 61/856,283, filed on Jul. 19, 2013, both of which are hereby incorporated by reference in their entireties.

### TECHNICAL FIELD AND BACKGROUND OF THE INVENTION

The present invention relates generally to the field of pulmonary instruments and, more particularly, to the field of devices and methods for the treatment of and prevention of pulmonary illnesses and diseases. The present invention is a pulmonary harmonica for achieving health and wellness through controlled breathing. The invention is a modified harmonica with specifically low-tuned reeds that produce frequencies not typically associated with normal or traditional harmonica musical performance that allows the user to generate a stream of controlled low frequency sound waves that loosen congestion in the user's lungs and sinuses resulting in improved breathing.

Pulmonary illnesses and diseases impact people around the world. People lose about half their lung capacity by age 70. It will be appreciated by those of ordinary skill in the art that such illnesses include asthma, bronchitis, emphysema, chronic obstructive pulmonary disease (COPD), and other bronchial, sinus, and airflow obstructing diseases. Such conditions may result from smoking, occupational exposures, air pollution, genetics, autoimmune diseases, acute exacerbations, and the like. People with such impaired lung function often have tenacious secretions that they cannot cough up and that worsen their condition. Additionally, many people, especially those overweight, have become habitual shallow breathers, which deprives them of sufficient oxygen for optimum health.

Traditional treatments have focused oral and intravenous medications as well as nebulizer and spacer administered medications. Despite the availability of such treatments for the conditions, respiratory muscle dysfunction frequently persists and worsens in patients.

Because of the muscle dysfunction, pulmonary patients are often referred to additional treatment in the form of pulmonary rehabilitation. Inspiratory and expiratory muscle training has sometimes been used instead of or in addition to such pulmonary rehabilitation. Such inspiratory muscle training has been recommended to be performed using a pressure or resistance device for a duration and frequency of up to 15-30 minutes and as often as 5-7 days per week. However, people do not enjoy using standard respiratory therapy devices because the devices are boring and clearly medical devices reminiscent of hospitals and the like. The monotony and relative frequency associated with such treatments may result in diminished effectiveness of treatments as patients may apply the treatments less often than recommended and for shorter durations than recommended. Likewise, patients may simply discontinue the treatments, being noncompliant with recommended self-care. Therefore, there is a need in the art for an effective pulmonary rehabilitation treatment using inspiratory and expiratory muscle training that improves the negative effects of muscle dysfunction and also encourages treatment regimen retention. There is also a need in the art for an effective pulmonary rehabilitation treatment using inspiratory and expiratory muscle training that loosens tenacious lung secretions and other fluids which can then be coughed up or otherwise eliminated from the body. There is also a need in the art for an effective pulmonary rehabilitation treatment using inspiratory and expiratory muscle training that promotes deep, abdominal breathing (i.e., diaphragmatic breathing). There is also a need in the art for an effective pulmonary rehabilitation treatment using inspiratory and expiratory muscle training that encourages continued use of the treatment device so that use of the device and the benefits of increased abdominal breathing both become habitual through practice. There is also a need in the art for an effective pulmonary rehabilitation treatment using inspiratory and expiratory muscle training that is smooth, attractive and pleasant to use, which empowers patients, although one with sharper edges would still be functional.

### SUMMARY OF THE INVENTION

It is therefore an object of the present invention to provide an effective pulmonary rehabilitation treatment using inspiratory and expiratory muscle training that improves the negative effects of muscle dysfunction and also encourages treatment regimen retention.

It is a further object of the present invention to provide an effective pulmonary rehabilitation treatment using inspiratory and expiratory muscle training that loosens lung secretions and other fluids that can then be more easily eliminated.

It is a further object of the present invention to provide an effective pulmonary rehabilitation treatment using inspiratory and expiratory muscle training that promotes long, slow, deep, and complete abdominal breathing.

It is a further object of the present invention to provide an effective pulmonary rehabilitation treatment using inspiratory and expiratory muscle training that encourages continued use of the treatment device so that use of the device and developed abdominal breathing both become habitual through practice.

It is a further object of the present invention to provide an effective pulmonary rehabilitation treatment using inspiratory and expiratory muscle training that is smooth, attractive and pleasant to use, which empowers patients, although such a device with sharper edges would be effective but not the optimal presentation of the device.

Further objects of the invention include helping people with respiratory disorders such as chronic obstructive pulmonary disease (COPD), asthma, chest or sinus congestion, or people with the desire for pulmonary therapy, to breathe better and enjoy life more. The invention is particularly designed to promote diaphragmatic breathing and produce deep, resonant, meditative sound waves that can be felt in the users' lungs but also penetrate to the sinuses. The invention requires no musical talent or ability whatsoever, and no prior experience with controlled breathing or meditative exercises and techniques. The invention is specifically designed to be smooth and comfortable to hold, with smooth edges that will prevent nicks and cuts in users with compromised immune systems or thinned skin in its optimal presentation.

These and other objects and advantages of the invention are achieved by providing a pulmonary harmonica device and method for using a pulmonary harmonica device. The device has low frequency notes that deliver powerful vibrations (pulses) to the user's respiratory system. When the method of the invention is practiced, as users blow and draw breaths through the low notes on this pulmonary harmonica, harmonic vibrations pulse the lungs and help loosen secretions that can be coughed up. This device promotes long, slow, deep, complete, abdominal breathing, which is continued after use because it becomes habitual through practice.

According to one embodiment of the invention, the device is a pulmonary harmonica for promoting deep abdominal breathing by a user having an outer housing, a comb having a mouthpiece portion and one or more holes, one or more reed plates, a plurality of reeds, located on the reed plates (unless the comb is designed to include that function), tuned to vibrate at low frequencies and to emit low frequency sound waves producing a harmonic resonance when blown or drawn by a user which pulses in the user's respiratory system resulting in the break-up of mucus, and fastening hardware for securing the comb, securing reeds to the reed plate (unless glued, melded, welded, brazed, or soldered) and the reed plate to the outer housing. In its extreme, the entire instrument could be molded or otherwise fashioned from fewer individual pieces, culminating in a single piece of material appropriately designed to deliver the same type of pulsing to the lungs from breathing through reeds.

According to another embodiment of the invention, the pulmonary harmonica may include that each one of the reeds is heavy, relative to reeds of a traditional harmonica in the same position (or hole) on the harmonica. This extra weight provides a variable resistance across several holes that helps build respiratory strength.

According to another embodiment of the invention, the pulmonary harmonica's lowest tuned reed has a frequency no higher than 150 Hz.

According to another embodiment of the invention, the pulmonary harmonica can be tuned to different tonal scales, such as having one or more reeds tuned to a frequency of 136.1 Hz or other chakra frequency below 150 Hz.

According to another embodiment of the invention, the comb may have 10 holes and the reeds may be further tuned to full diatonic scales of chords in a major or minor key.

According to another embodiment of the invention, the comb may include a single and only a single hole, the one or more reed plates may include a single and only a single reed plate, and the plurality of reeds may include two and only two reeds.

According to another embodiment of the invention, the outer housing may include two smooth cover plates.

According to another embodiment of the invention, the smooth cover plates, comb, and the reeds further may include anti-microbial materials for improved hygienic function.

According to another embodiment of the invention, the fastening hardware may be selected from the group comprising screws, bolts, rivets, nails, welds, melds, and glues.

According to another embodiment of the invention, the pulmonary harmonica may include a built-in force guide which elicits a buzzing sound from one of the reeds when the device is used with excessive force during inhalation by the user.

According to another embodiment of the invention, the pulmonary harmonica may include a built-in force guide which elicits a buzzing sound from one of the reeds when the device is used with excessive force during exhalation by the user.

According to another embodiment of the invention, the device may be tuned in order to provide a pleasant sound in its optimal presentation. The device is smooth, attractive and pleasant to use, which empowers patients. The present invention may be characterized by lack of a discordant sound which is present and heard in traditionally tuned harmonicas. Thus, the present invention is even more suitable for people with no musical ability. Use of the harmonica device of the present invention may motivate patients to continue to use the treatment. The device may provide strong vibrations to the lungs. Rather than having sharp edges that can cut fragile skin, the optimal presentation of the device has an exterior surface that is smooth, although some presentations of the device could have sharp edges and still develop effective low frequency pulses. The optimal presentation of the device may be considered attractive and pleasant to use.

According to an embodiment of the invention, the device may include a comb, reeds, some of which may have weights on the end, reed plate(s), cover plate(s), and fastening hardware such as screws.

According to another embodiment of the invention, there may be two cover plates—upper and lower, two reed plates with reeds attached, a central comb with holes for mouthpiece and air movement, and fasteners to attach the reeds to the reed plates and secure all components together.

According to another embodiment of the invention, the device may include as few as one sounding hole and one reed plate, as long as there are at least two reeds tuned to resonant frequencies, and could be square, rectangular, cylindrical, curved or other overall shape in appearance.

According to another embodiment of the invention, any number of sounding holes may be included. Ten holes allows for two full diatonic scales, including scales of chords in key, which may provide a wide range of resistance training and may be tuned to allow the user to play songs.

According to another embodiment, the number of screws used to fasten the reed plates to the comb can vary. The device parts may be fastened from a single side using tapped holes on the opposing reed plate for fastening or parts could be nailed, welded, glued, or otherwise fastened together.

According to another embodiment of the invention, the device may be tuned to different frequencies. According to one such embodiment, some reeds may be tuned to a specific frequency that is not a standard musical note, such as 136.1 Hz, which is commonly acknowledged to be the frequency of the mystical Sanskrit symbol OM and the Hindu heart Chakra. The invention specifically contemplates tuning the reeds to various Chakra frequencies. In tuning the device in this way, the device will make a sound that can be highly meditative or evocative to followers of Hinduism, Buddhism, Sikhism, and Jainism, as well as to people who meditate without a specific affiliation.

According to another embodiment of the invention, the upper limit on the lowest frequency reed for the invention may be 150 Hz. Further, optimal pulsing can be achieved for most people by using a range of single reed frequencies below 150 Hz as the lower notes and combining them as chords. Using multiple reeds below 150 Hz further optimizes the usefulness of the invention for more people by offering a user-selectable amount of pulsing for their lungs. For convenience, the pulsing effect can be generated by the invention when reduced to as few as one hole and two reeds tuned to resonant frequencies.

According to another embodiment of the invention, the cover plates may be configured with a relatively low profile. Such a configuration may include a built-in force guide which elicits a buzzing of one of the reeds when the device is used with excessive force during inhalation on low chords.

According to another embodiment of the invention, the cover plates may be configured with a relatively low profile. Such a configuration may include a built-in force guide which elicits a buzzing of one of the reeds when the device is used with excessive force during exhalation on low chords.

According to another embodiment of the invention, the device may encourage longer reed life by limiting the travel of the lower pitched reeds.

According to another embodiment of the invention, the combs, cover plates, or reeds may be manufactured with anti-microbial materials, such as anti-microbial polymers, coatings, ceramics, copper and its alloys (brasses, bronzes, cupronickel, copper-nickel-zinc, and others), silver, or stainless steel. As such, the hygienic function may be improved for some conditions.

According to a method of practicing the invention, the device may be held by the user in either one or both hands by the cover plate(s). According to another embodiment, a supportive device may be used in lieu of hands. The comb or hole(s) may be held up to the user's mouth. The user may then blow and/or draw breath through the comb in a controlled, comfortable manner. The airflow created by the user's lungs across the reeds generates low frequency sound waves. These low frequency sound waves vibrate in the user's respiratory system, resulting in the break-up of mucus and congestion in the user's lungs and sinuses. These effects can be achieved extremely quickly, sometimes in just a few minutes. With regular use, the user is able to breathe clearer, feel better, and experience an improved quality of life. The beneficiaries of improved oxygenation include patients who have decreased lung function and those who are recovering from surgery or trauma, or other acute diseases, long-term wound patients, including diabetics, people whose obesity has restricted their lung function, people with panic attacks and other mood disorders, dementia, chronic fatigue, asthma, and sinus or lung congestion. Meditation is known to have a wide range of beneficial side effects, including a calmer state of mind which is especially important when dealing with the inability to breathe or an impending asthma attack.

According to another embodiment of the method, musicians and athletes could use this device to build lung capacity to improve performance. People who meditate could use this device as a meditation aide. People with mild respiratory distress or acute respiratory illness could use this device to help loosen secretions and relax. People interested in improving the oxygenation of their body may use this device to derive the benefits of deep abdominal breathing against variable resistance. By encouraging meditation, the device can help lower blood pressure and stress hormone levels and the resulting inflammation. The meditative aspect combined with giving people something to do with their hands and mouth could also make this device a smoking cessation or weight loss aide.

### BRIEF DESCRIPTION OF THE DRAWING FIGURES

The present invention is best understood when the following detailed description of the invention is read in view of the accompanying drawings.

FIG. 1 is an environmental perspective view of an embodiment of the pulmonary harmonica device with the device tipped upwards to make playing single notes easier;

FIG. 2 is an environmental perspective view of an embodiment of the pulmonary harmonica device with the device held straight-in to promote deep abdominal breathing consistent with the method of the present invention;

FIG. 3 is an exploded perspective view of an embodiment of the pulmonary harmonica device having ten holes and twenty reeds;

FIG. 4 is a partial exploded perspective view of an embodiment of the pulmonary harmonica device highlighting the reed structure;

FIG. 5 is a side view of an embodiment of the pulmonary harmonica device;

FIG. 6 is a top view of an embodiment of the pulmonary harmonica device;

FIG. 7 is a an end view of an embodiment of the pulmonary harmonica device;

FIG. 8 is an exploded perspective view of an embodiment of the pulmonary harmonica device having two holes and four reeds;

FIG. 9 is an exploded perspective view of and embodiment of the pulmonary harmonica device having one hole and four reeds;

FIG. 10 is an exploded perspective view of an embodiment of the pulmonary harmonica device having one hole and two reeds;

FIG. 11 is a perspective view of an embodiment of the pulmonary harmonica device having two holes; and

FIG. 12 is a perspective view of an embodiment of the pulmonary harmonica device having one hole.

### DETAILED DESCRIPTION OF THE PREFERRED EMBODIMENT

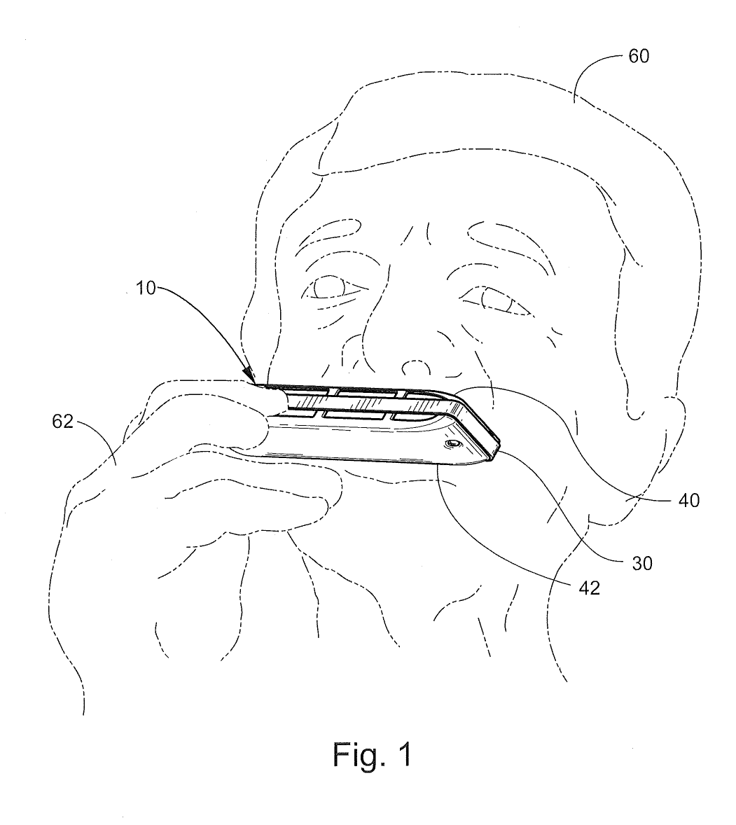

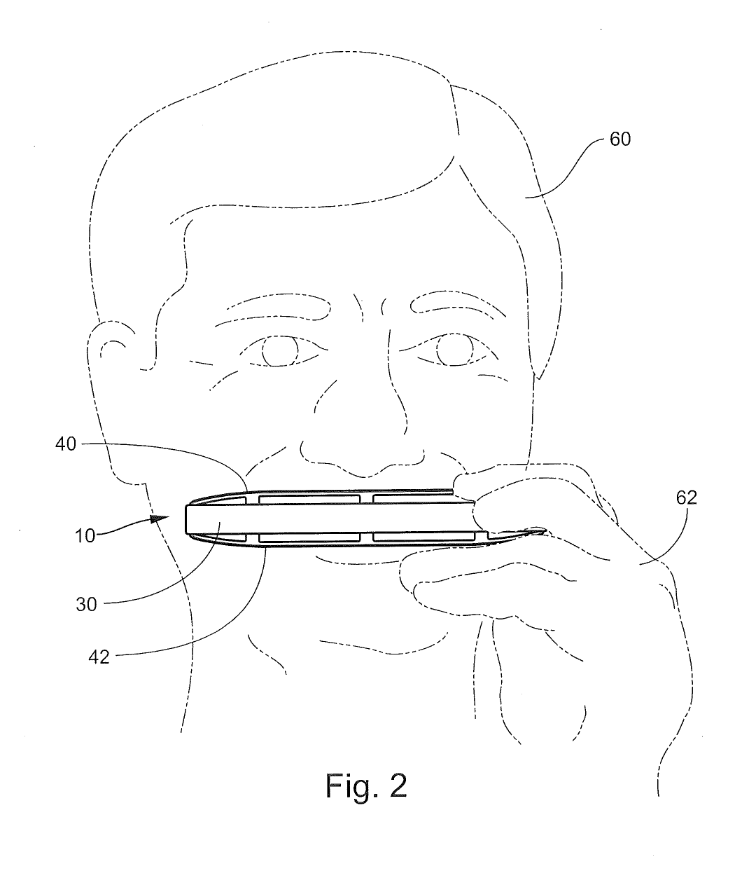

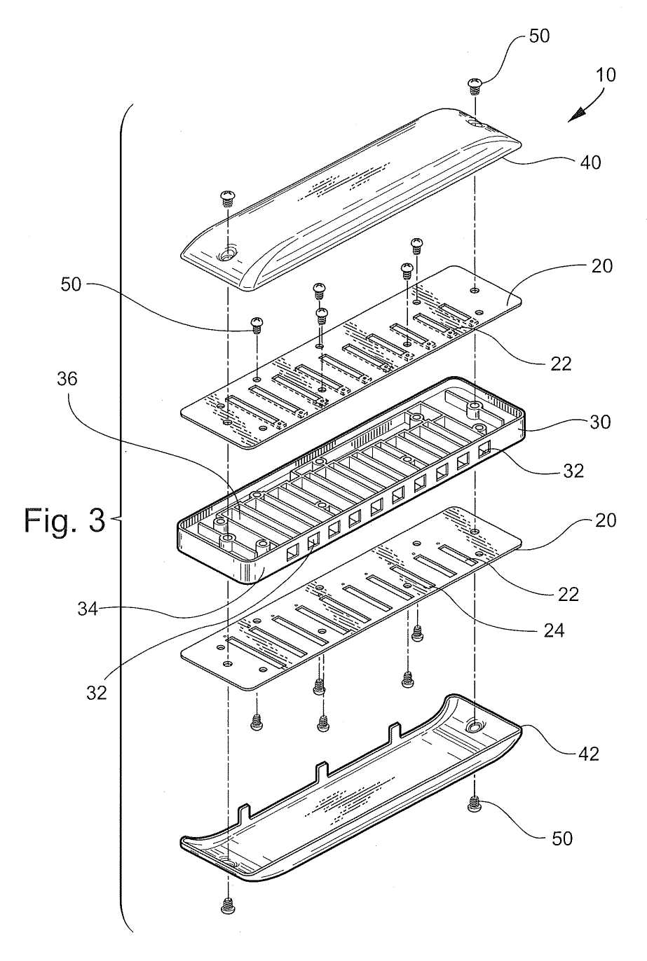

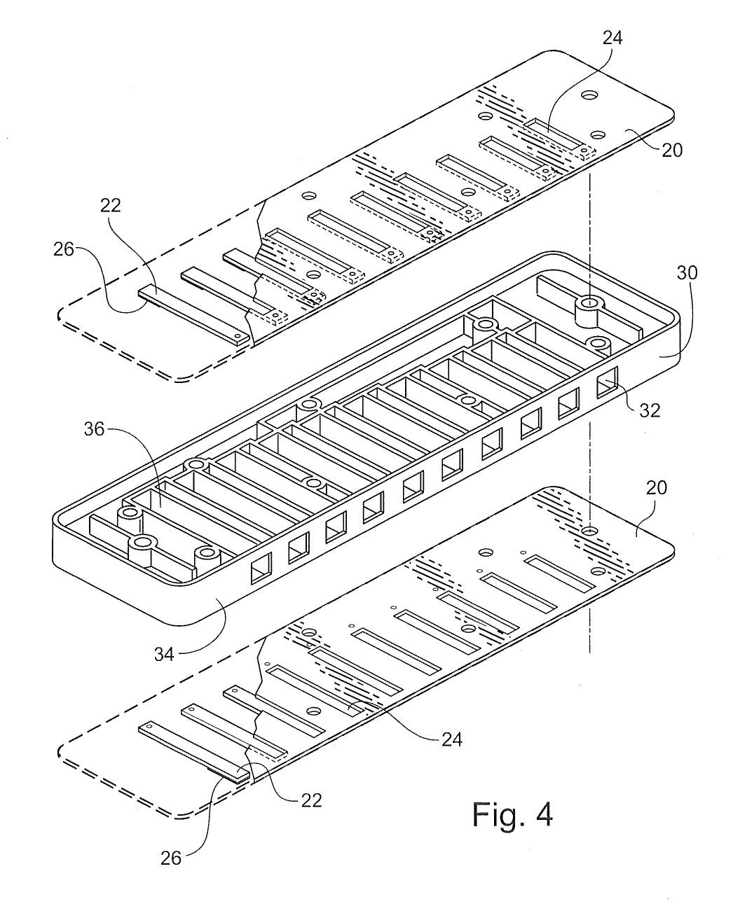

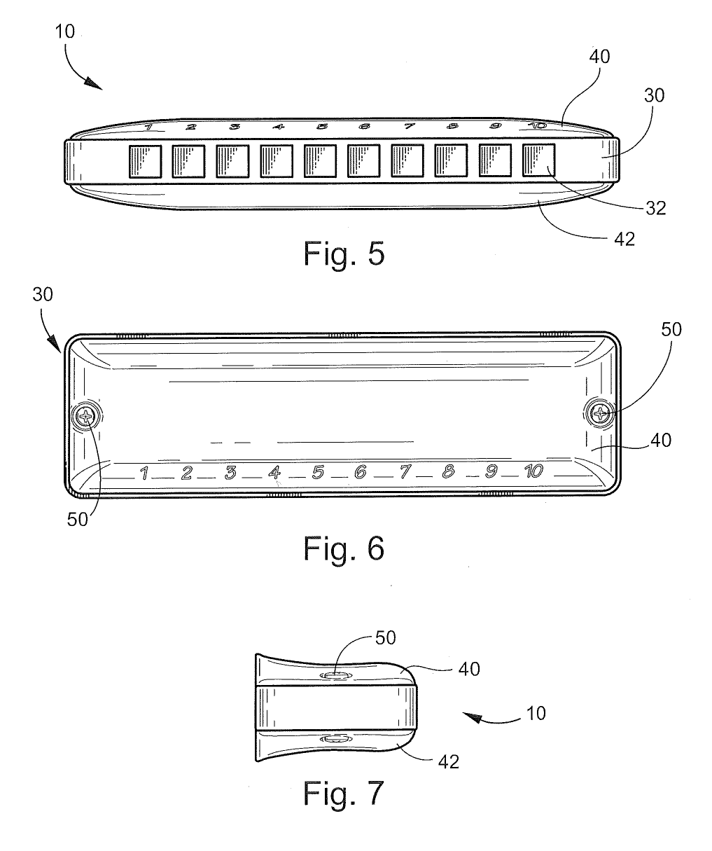

Referring now specifically to the drawings, a pulmonary harmonica device is shown generally at reference numeral 10. As shown in FIGS. 1 and 2, the device 10 and method, which is especially shown in FIG. 2, of the present invention can be used by anyone, just about anywhere, without musical talent or experience with musical instruments, and with no prior knowledge or experience with meditation or controlled breathing exercises and techniques. As shown in FIGS. 3 and 4, the parts of the pulmonary harmonica device 10 include a comb 30 or other structure to hold the reeds 22 in place, two or more reeds 22, one or more reed plates 20 or other structure to allow for a gap through which the reed 22 passes in use, a housing in the form of one or more cover plates 40, 42, fastening hardware 50, and low frequency sound waves. The reed plate 20 is defined as a structure to allow for a gap through which the reed 22 passes in use. The reeds 22 may have weights 26 attached the ends of the reeds 22.

The cover plates 40, 42 and comb 30 can be made of plastic, wood, brass, aluminum, bronze, or stainless steel, or other materials with sufficient integrity (with or without antimicrobial properties), and can be plated, painted, lacquered, shellacked, oiled, or anodized. The reeds 22 and reed plates 20 can be made of brass, bronze, German silver, stainless steel, aluminum, plastic, titanium, or other material with appropriate spring-like characteristics. Alternatively, it may be molded out of a single piece of plastic, ceramic, or like materials. The tuning of the reeds 22 can be in any key with two restrictions: low-pulse generating methodology is followed (i.e., reeds tuned to resonant frequencies, such as the notes that form a major or minor chord), and the pulses generated are sufficiently low to be felt in the upper-chest. Although people's sensitivities to chest pulsing varies, harmonicas meeting the criteria for this invention and pragmatically low enough in frequency to be effective will need to have their lowest tuned reed of the reeds 22 no higher than 150 Hz.

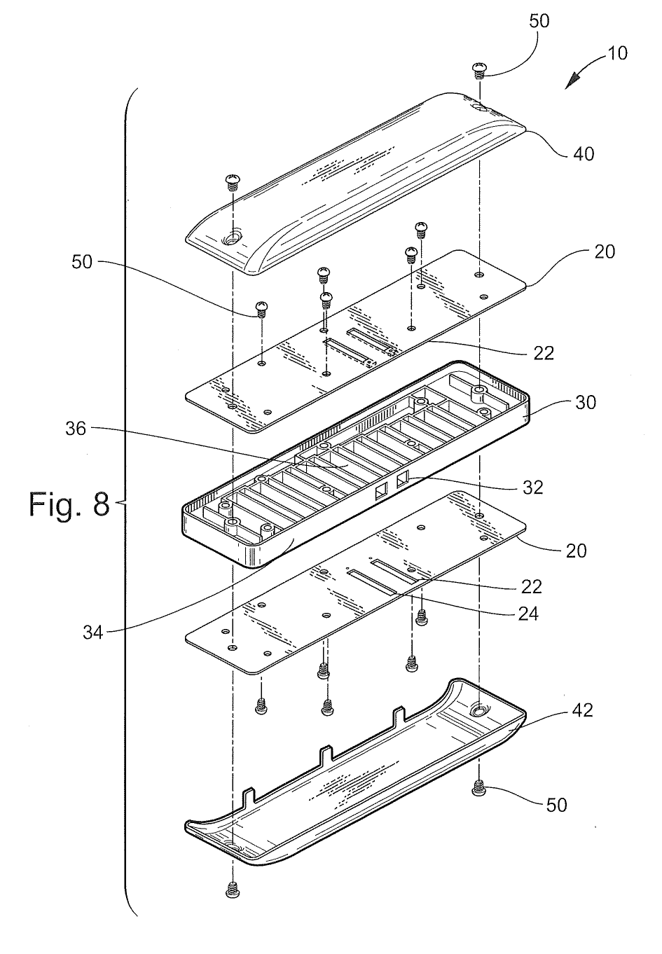

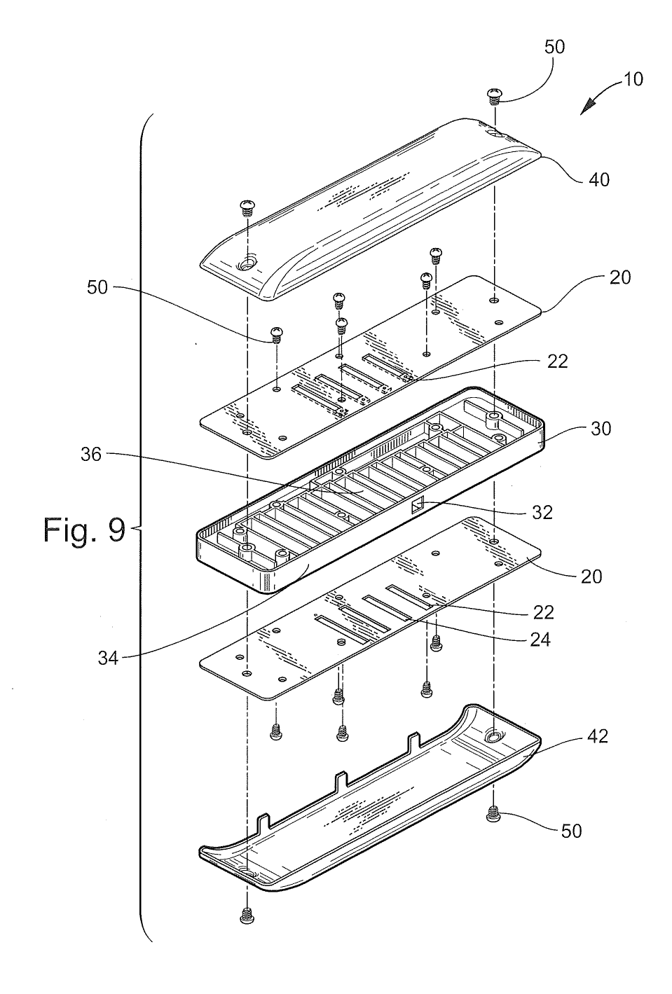

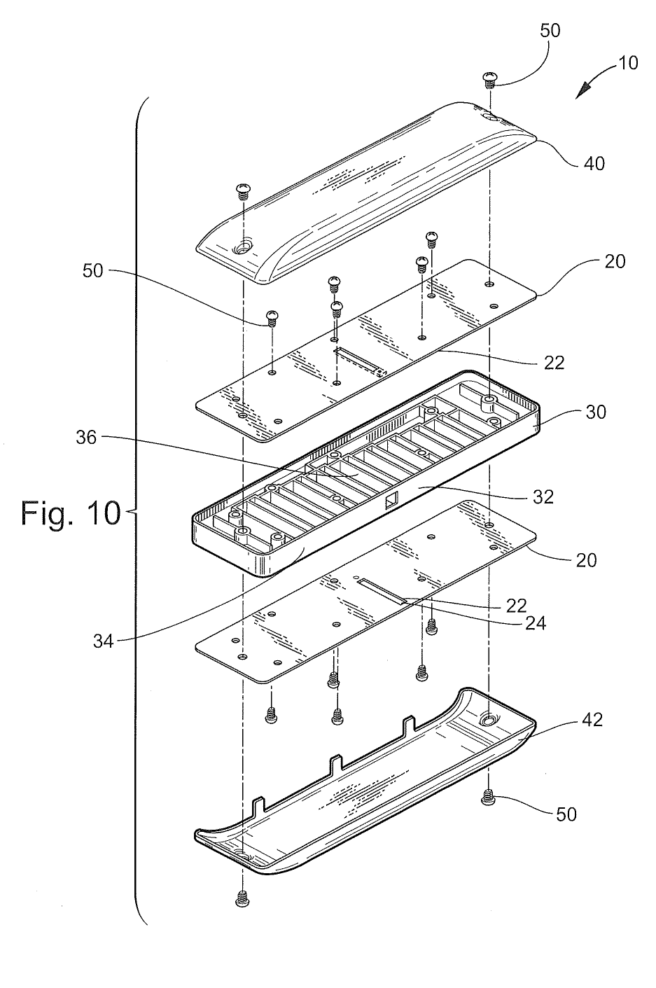

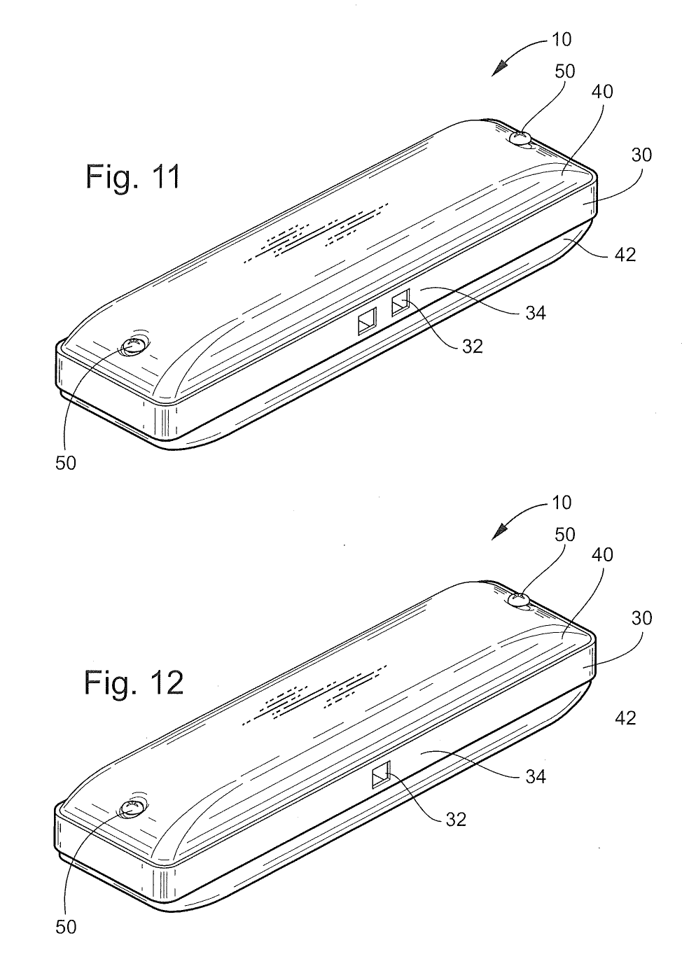

The device 10 is used by blowing or drawing air through one or more holes 32 along the mouthpiece portion 34 of the comb 30. Behind the holes are chambers 36 containing at least one reed 22 which may be attached to a reed plate 20. The reed 22 is a flat elongated spring typically made of brass, bronze, or stainless steel, which is secured at one end over a slot 24 that serves as an airway. When the free end of the reed 22 is made to vibrate by blowing or drawing breath across it, it blocks and unblocks the airway to produce sound and a resistance that varies with pitch. By tuning the reeds 22 to vibrate at specific low frequencies, a harmonic resonance is set up, which pulses the lungs of a user 60 and helps loosen secretions and enable expectoration. Low-tuned reeds 22 are heavy and hard to blow and draw relative to a traditional harmonica. These low-tuned reeds 22 provide a resistance that helps build respiratory strength. By having some reeds 22 tuned higher and others lower, variable resistance training can be accomplished. By having the reeds 22 tuned to full diatonic scales of chords in its major or minor key, dissonant notes and chords are eliminated and the improved pulmonary harmonica 10 can be used by anyone regardless of musical background or ability. Ten-hole tuning as shown in FIG. 3, can allow two octaves of the complete diatonic scale of chords, and includes spiral tuning that can be used for a wide variety of songs. Fewer than 10 holes, as in FIGS. 8-12, can also be used with this spiral tuning and a full octave can still be achieved. By having smooth edges on all surfaces of the device 10 and only pleasant sounds, the device is safe and encourages compliance. Because the device 10 makes noise, it is easy to monitor compliance by a user 60. The draw and blow notes can also be tuned to as few as one extended chord so that people using the device 10 in a group can all sound harmonious when using the device together and with no knowledge of music or what to play.

The device 10 is intended for use by people 60 with respiratory disorders or who seek the benefits of improved oxygenation and meditative relaxation. As shown in FIG. 1 and FIG. 2, the device 10 is held by the user 60 in either one or both hands 62 by the cover plate(s) 40, 42 or by a holder designed for the purpose. The comb 30 and/or blow/draw hole(s) 32 is held up to the user's 60 mouth. The user 60 then blows and/or draws breath through comb 30 in a controlled, comfortable manner. Preferably, the device 10 is utilized as in FIG. 2 with the device 10 held straight-in relative to the mouth of the user 60 to promote deep abdominal breathing. The airflow created by the user's 60 lungs across the reeds 22 generates low frequency sound waves. These low frequency sound waves vibrate in the user's 60 respiratory system, resulting in the break-up of mucus and congestion in the user's 60 lungs and sinuses. The invention 10 contemplates a regimen of controlled breathing using the device. These effects can be achieved extremely quickly. With regular use of the device 10, the user 60 is able to breathe clearer, feel better, and experience an improved quality of life.

A pulmonary harmonica device 10 and method for using a pulmonary harmonica device 10 according to the invention has been described. Various details of the invention may be changed without departing from the scope of the invention. Furthermore, the foregoing description of the preferred embodiments of the invention and best mode for practicing the invention are provided for the purpose of illustration only and not for the purpose of limitation, the invention being defined by the claims.

We claim:

1. A pulmonary harmonica device for promoting deep abdominal breathing by a user and for producing a sound comprising:
(a) an outer housing;
(b) a comb having a mouthpiece portion and one or more holes;
(c) one or more reed plates;
(d) a plurality of reeds, located on the reed plates, tuned to vibrate at low frequencies and to emit low frequency sound waves producing a harmonic resonance when blown or drawn by a user which pulses in the user's respiratory system resulting in the break-up of mucus; and
(e) fastening hardware for securing the reeds, the reed plates, the comb, and the cover plates together.

2. The pulmonary harmonica device of claim 1 wherein each one of the plurality of reeds are heavy, relative to reeds of a traditional harmonica, for providing a resistance that helps build respiratory strength.

3. The pulmonary harmonica device of claim 2 wherein a lowest tuned reed of the plurality of reeds is no higher than 150 Hz.

4. The pulmonary harmonic device of claim 3 wherein one or more reeds is tuned to a Chakra frequency.

5. The pulmonary harmonica device of claim 3 wherein the comb has 10 holes, the reeds are further tuned to full diatonic scales of chords in a major key or a minor key, and the sound produced is characterized by a lack of discordant sound.

6. The pulmonary harmonica device of claim 3 wherein the comb comprises a single and only a single hole, the one or more reed plates comprises a single and only a single reed plate, and the plurality of reeds comprises two and only two reeds.

7. The pulmonary harmonica device of claim 3 wherein the outer housing comprises two smooth cover plates.

8. The pulmonary harmonica device of claim 7 wherein the comb, the smooth cover plates, and/or the reeds further comprise anti-microbial materials for improved hygienic function.

9. The pulmonary harmonica device of claim 8 wherein fastening hardware is selected from the group comprising screws, bolts, nails, welds, rivets, melds, and glues.

10. The pulmonary harmonica device of claim 8 wherein the housing, the comb, the reed plates, and the reeds are molded from a single piece of material.

11. The pulmonary harmonica device of claim 7 further comprising a built-in force guide which elicits a buzzing sound from one of the reeds when the device is used with excessive force during inhalation and/or exhalation by the user.

12. A method of using a pulmonary harmonica device to treat a patient having a respiratory disorder comprising the steps of:
(a) providing the pulmonary harmonica to the patient, the pulmonary harmonica comprising:
i. an outer housing;
ii. a comb having a mouthpiece portion and one or more holes;
iii. one or more reed plates; and
iv. a plurality of reeds, located on the reed plates, tuned to vibrate at low frequencies and to emit low frequency sound waves producing a harmonic resonance when blown or drawn by the patient, wherein a lowest tuned reed of the plurality of reeds is no higher than 150 Hz;
(b) placing hands on the housing by the patient or utilizing a supportive device and holding the device at the mouth of the patient;
(c) blowing through the holes by the patient in a controlled comfortable manner to produce low frequency sound waves and to produce harmonic vibrations which pulse the patient's lungs and sinuses;
(d) drawing through the holes by the patient in a controlled comfortable manner to produce low frequency sound waves and to produce harmonic vibrations which pulse the patient's lungs and sinuses;
(e) repeating steps (c) and (d).

13. The method of using a pulmonary harmonica device to treat a patient having a respiratory disorder of claim 12 wherein the respiratory disorder is selected from the group consisting of: chronic obstructive pulmonary disease (COPD), asthma, chest congestion, sinus congestion, bronchitis, and emphysema.

14. The method of using a pulmonary harmonica device to treat a patient having a respiratory disorder of claim 13 wherein each one of the plurality of reeds of the pulmonary harmonica provided in step (a) are heavy, relative to reeds of a traditional harmonica, for providing a resistance that helps build respiratory strength.

15. The method of using a pulmonary harmonica device to treat a patient having a respiratory disorder of claim 13 wherein one or more reeds is tuned to a Chakra frequency.

16. The method of using a pulmonary harmonica device to treat a patient having a respiratory disorder of claim 13 wherein the comb of the pulmonary harmonica provided in step (a) has 10 holes, the reeds are further tuned to full diatonic scales of chords in a major or a minor key, and the sound produced is characterized by a lack of discordant sound.

17. The method of using a pulmonary harmonica device to treat a patient having a respiratory disorder of claim 13 wherein the comb of the pulmonary harmonica provided in step (a) comprises a single and only a single hole, the one or more reed plates comprises a single and only a single reed plate, and the plurality of reeds comprises two and only two reeds.

18. A method of using a pulmonary harmonica device to meditate and to improve oxygenation of a user's body comprising the steps of:
(a) providing the pulmonary harmonica to a user, the pulmonary harmonica comprising:
i. an outer housing;
ii. a comb having a mouthpiece portion and one or more holes;
iii. one or more reed plates; and
iv. a plurality of reeds, located on the reed plates, tuned to vibrate at low frequencies and to emit low frequency sound waves producing a harmonic resonance when blown or drawn by the patient, wherein one or more reeds tuned to a Chakra frequency and a lowest tuned reed of the plurality of reeds is no higher than 150 Hz;
(b) placing hands on the housing by the user or utilizing a supportive device and holding the device near the mouth of the user;
(c) blowing through the holes by the user in a controlled comfortable manner to produce low frequency sound waves and to produce harmonic vibrations which pulse the patient's lungs and sinuses;
(d) drawing through the holes by the user in a controlled comfortable manner to produce low frequency sound waves and to produce harmonic vibrations which pulse the patient's lungs and sinuses;
(e) repeating steps (c) and (d).

19. The method of using a pulmonary harmonica device to meditate and to improve oxygenation of a user's body of claim 18 wherein each one of the plurality of reeds of the pulmonary harmonica provided in step (a) are heavy, relative to reeds of a traditional harmonica, for providing a resistance that helps build respiratory strength.

20. The method of using a pulmonary harmonica device to meditate and to improve oxygenation of a user's body of claim 18 wherein the comb of the pulmonary harmonica provided in step (a) has 10 holes.

21. The method of using a pulmonary harmonica device to meditate and to improve oxygenation of a user's body of claim 18 wherein the comb of the pulmonary harmonica provided in step (a) comprises a single and only a single hole, the one or more reed plates comprises a single and only a single reed plate, and the plurality of reeds comprises two and only two reeds.
